(19) United States
(12) Patent Application Publication
(10) Pub. No.: US 2023/0259500 A1
(43) Pub. Date: Aug. 17, 2023

(54) DATA STORAGE AND RETRIEVAL

(71) Applicant: GEMVIZ, LLC, Winona Lake, IN (US)

(72) Inventors: Jody L. Claypool, Warsaw, IN (US); Vinay Tikka, Winona Lake, IN (US)

(21) Appl. No.: 18/110,264

(22) Filed: Feb. 15, 2023

Related U.S. Application Data
(60) Provisional application No. 63/310,227, filed on Feb. 15, 2022.
(60) Provisional application No. 63/310,280, filed on Feb. 15, 2022.

(51) Int. Cl. G06F 16/22; G06F 16/245

(52) U.S. Cl. CPC G06F 16/2228; G06F 16/245

(57) ABSTRACT

A data structure of a ledger file includes an object identifier that identifies the ledger file for an asset related to a physical entity; a digital model of the asset; attributes, each attribute corresponding to a detail associated with the asset related to the physical entity and having properties providing a record of information about the asset or the detail associated therewith; and an updates index for storing security tags, each security tag having information including a block hash and a timestamp. A data storage and retrieval system for one or more computers includes a ledger file generator that generates a data structure of a ledger file in the one or more computers and an updater that assigns a corresponding security tag to an updates index for indexing each newly added record of information of an attribute to the ledger file.

### CROSS-REFERENCE TO RELATED APPLICATION(S)

This application claims the benefit of U.S. Provisional Application Ser. No. 63/310,227, filed Feb. 15, 2022, and U.S. Provisional Application Ser. No. 63/310,280, filed Feb. 15, 2022.

### BACKGROUND

Virtual reality describes an immersive simulated experience. Mixed reality and augmented reality combine digital/virtual renderings with the real world. Items in a virtual reality environment (whether completely immersive or in a mixed/augmented form) may be designed to appear as realistic as possible or, even if presented as something not possible or that does not exist in the real world, have features and functionality that people are familiar with from the real world. With the continued interest in creating a “metaverse” where people can communicate with each other, perform activities, and conduct transactions in a virtual and augmented reality environment, there is a need for technologies that can support the association between the real physical entity (e.g., person, thing) and the virtual and/or digital representation of that entity, especially as things change with respect to the real physical entity over time.

### BRIEF SUMMARY

Systems and methods providing data storage and retrieval are described. A data structure is presented that supports the association of an asset in real-space to a virtual/digital form as things change with the asset over time, for example, for the life of the asset. The association can be performed synchronously such that as information is collected about the asset (e.g., by sensors, manual entry of information, etc.), the virtual/digital information is also updated.

A data structure of a ledger file is provided that includes an object identifier that identifies the ledger file for an asset related to a physical entity; a digital model of the asset; attributes, each attribute corresponding to a detail associated with the asset related to the physical entity and having properties providing a record of information about the asset or the detail associated therewith; and an updates index for storing security tags, each security tag having information including a block hash and a timestamp.

A data storage and retrieval system for one or more computers includes a ledger file generator that generates a data structure of a ledger file in the one or more computers and an updater that assigns a corresponding security tag to an updates index for indexing each newly added record of information of an attribute to the ledger file.

A record retriever can be included that can enable reading and access of blocks of data using a corresponding security tag.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIGS. 1A and 1B illustrate conceptual representations of a ledger file.

FIGS. 2A and 2B are block diagram that describe example data storage and retrieval systems according to some embodiments of the present disclosure.

FIG. 3 is a flowchart that describes a method for storing and retrieving data from one or more computers, according to some embodiments of the present disclosure.

FIG. 4 is a flowchart that describes a further method for storing and retrieving data from one or more computers, according to some embodiments of the present disclosure.

### DETAILED DESCRIPTION

Systems and methods providing data storage and retrieval are described.

The detailed descriptions which follow are presented largely in terms of algorithms and symbolic representations of operations on data bits within memory/computers. These descriptions and representations are the means used by those skilled in the data processing arts to most effectively convey the substance of their work to others skilled in the art.

An algorithm is here, and generally, conceived to be a self-consistent sequence of steps leading to a desired result. These steps are those requiring physical manipulations of physical quantities. Usually, though not necessarily, these quantities take the form of electrical or magnetic signals capable of being stored, transferred, combined, compared, and otherwise manipulated. It proves convenient at times, principally for reasons of common usage, to refer to these signals as bits, values, elements, symbols, characters, terms, numbers, or the like. It should be borne in mind, however, that all of these and similar terms are to be associated with the appropriate physical quantities and are merely convenient labels applied to these quantities.

Further, the manipulations performed are often referred to in terms, such as adding or comparing, which are commonly associated with mental operations performed by a human operator. No such capability of a human operator is necessary, or desirable in most cases, in any of the operations described herein which form part of the present invention; the operations are machine operations. Useful machines for performing the operations of the present invention include general purpose digital computers or other similar digital devices. In all cases there should be borne in mind the distinction between the method operations in operating a computer and the method of computation itself. Method steps are directed to operating a computer in processing electrical or other (e.g., mechanical, chemical) physical signals to generate other desired physical signals.

Certain embodiments may be implemented as a computer process, a computing system, or as an article of manufacture, such as a computer program product or computer-readable storage medium. Certain methods and processes described herein can be embodied as software, code and/or data, which may be stored on one or more storage media. Certain computer program products may be one or more computer-readable storage media readable by a computer system (and executable by a processing system) and encoding a computer program of instructions for executing a computer process. It should be understood that as used herein, in no case do the terms “storage media”, “computer-readable storage media” or “computer-readable storage medium” consist of transitory carrier waves or propagating signals.

Any systems or apparatus described herein may be specially constructed for the required purposes or it may comprise a general-purpose computer as selectively activated or reconfigured by a computer program stored in the computer. The algorithms presented herein are not inherently related to a particular computer or other apparatus. In particular, various general-purpose machines may be used with programs written in accordance with the teachings herein, or it may prove more convenient to construct more specialized apparatus to perform the required method steps. The required structure for a variety of these machines will appear from the descriptions given below.

A ledger file format, referred to herein as an EOX file format, is used to track changes/updates to a digital model/asset in its entire life cycle, following the changes and updates of the corresponding physical asset and/or physical entity to which the asset is related. An asset is a useful or valuable thing, person, or quality. A physical entity is a thing with a distinct and independent existence, which has a presence that is tangible. An asset related to a physical entity is not required to be of material existence and can include concepts and other abstractions. The described EOX file format can be used to support the association between the real physical asset and the virtual representation of that asset as things change over time.

Through a data storage and retrieval system that generates a data structure of a ledger file, it is possible for digital assets to track various attributes of the physical world representation of those digital assets. As the physical asset/physical world representation goes through its life cycle (e.g., from manufacturing to utilization to retirement), the ledger file not only tracks any changes to the attributes but also maintains the validity of the changes through an update index of the ledger file.

Application of the EOX file and its tracing data through the life cycle of an asset is helpful for medical devices, implants, pharmaceuticals, nanoceuticals, sports gear, and many other physical entities.

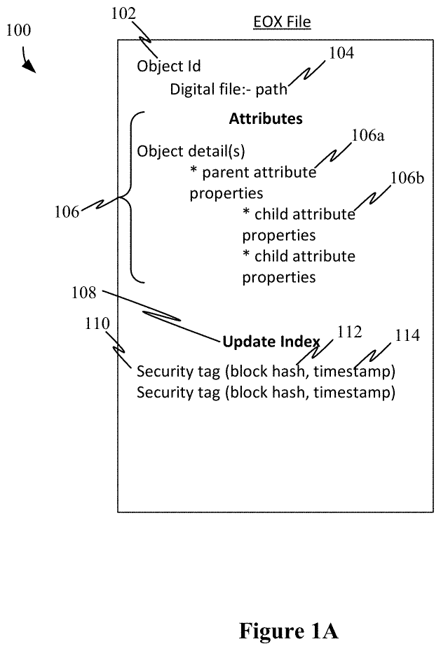

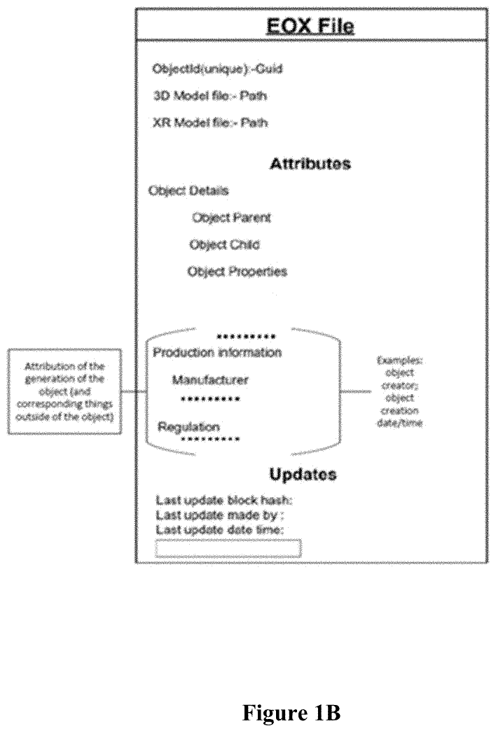

FIGS. 1A and 1B illustrate conceptual representations of a ledger file.

Referring to FIG. 1A, a data structure of a ledger file 100 includes an object identifier 102 that identifies the ledger file for an asset related to a physical entity; a digital model 104 of the physical object; attributes 106, each attribute corresponding to a detail associated with the asset related to the physical entity and having properties providing a record of information about the asset or the detail associated therewith; and an updates index 108 for storing security tags 110, each security tag having information including a block hash 112 and a timestamp 114.

The object identifier 102 identifies the ledger file for an asset related to a physical entity in the physical world. This identifier can be considered a routing number that associates the virtual asset with its respective asset in the physical world. The object identifier 102 represents the parent entity to which attributes can be applied (e.g., the top most element to which any other assets and attributes can be added to or built from). Conceptually, the object identifier can be considered to identify a primary block from which next blocks are added or a root of a tree from which next nodes are connected. A block can be defined as a set/collection of things (e.g., objects, transactions, etc.), where the size is predefined.

The digital model 104 of the asset can be a 3-D model file, a virtual reality file (compatible with virtual, augmented, mixed, and/or extended virtual reality), an image file, a document file, and the like. In some cases, the ledger file 100 includes the digital model 104 in the form of resource location information (e.g., a file path) of the digital model. In some cases, multiple digital models may be included, for example, of different file types for different visual environments (e.g., virtual reality, augmented reality, conventional display screens).

An attribute 106 corresponds to a detail associated with the asset related to the physical entity. The association may be based on a direct relationship, for example, based on any form of information about the physical entity. In some cases, a loose association of attributed data can be included, allowing for additional connections between entities. Examples of loosely associated attributes include community information (e.g., information from other similar physical entities or other users who have acquired similar entities), funding and regulation information (e.g., insurance/government regulation), and other aspects that may of interest for filtering and sorting of information. Attributes have properties (and in some cases certain properties can be considered attributes).

Attributes 106 can include a parent attribute 106a indicating a source of the information or indicative of a category of information; and one or more child attributes 106b of the parent attribute, the one or more child attributes being related to content associated with the source of information or being indicative of an entity in the category of information.

An attribute 106 (e.g., parent attribute 106a or child attribute 106b) can indicate the source of the information, for example, an application or device from which information about the asset and/or physical entity is received or derived.

Attributes 106 can include attribution information indicating a source or creator of the asset related to the physical entity and/or the physical entity itself.

In addition to a block hash 112 and time stamp 114, a security tag 110 can further include what or who is a source of the update. In some cases, the source of the update is an application or device. In some cases, the source of the update indicates the attribute that is updated, for example, when the attribute is an application or device.

The ledger file 100 thus can be composed of blocks where each block stores data about the asset related to the physical entity and its attributes.

Referring to FIG. 1B, in an illustrative scenario, when generating the EOX file, the asset related to a physical entity is given a unique identifier (ObjectId) for the EOX file and can include content such as a 3D model file (e.g., CAD file), virtual reality file, image file, document file, etc. In this manner, the initial generation of the EOX file creates a first block having the ObjectId and containing the content. The EOX file can include the location (i.e., path) information for the content or be stored with content/digital model itself. Here, the content supports the physical entity to which the asset relates. For example, the asset can be a person which will be represented as an avatar and the physical entity can be a specific person. In such a case, the content is a 3D model file and/or a virtual reality (XR) file of the specific person used to generate the avatar of the specific person. As another example, the asset related to the physical entity can be an asset related to a medical device. The asset can be considered a concept or other abstraction holding space for a medical device (e.g., to capture the information and concepts of a life cycle of a medical device from design through use and end of use). In such a case, the content can include a CAD file of the medical device (or a file path to the CAD file) generated during a design stage of the medical device, providing a virtual representation of the medical device. Where there is a virtual reality file format representation, the virtual reality file or file path to the virtual reality file can also be included.

Attributes of the asset can be different based on the entity to which the asset relates. For example, a parent attribute may reference an Apple Watch® that the person represented by the asset wears. The children and properties of that parent attribute can include applications and data collected by the Apple Watch® (e.g., steps, heart rate, etc.).

Attributes providing attribution information and even regulatory frameworks/policy information (e.g., information related to but may not be a direct detail) can be part of the metadata of the file as well. For example, with an asset related to a medical device, the attribution information associated with the EOX file can include product information such as manufacturer and regulation/regulatory framework information.

As explained above, the EOX file accumulates data about the asset related to the physical entity and/or physical entity (i.e., as things occur in the real world, the virtual/digital asset is updated to reflect those things). A security tag associated with the data is added to the ledger for each update/accumulated data. The security tag is generated, for example, as a block hash, and with a time stamp and, in some cases, identity of the updater source (e.g., the updating application or device—which may be indicated by the attribute). The security tag acts as a unique key to access the data of an update. This allows for the content to be viewed and otherwise accessed by an application. The file format is suitable for viewing/interacting in virtual reality environments as well as conventional 2-D (e.g., by a graphical user interface of an application).

The organizational structure illustrated in FIGS. 1A and 1B are for conceptual purposes and the content of the EOX file may be stored in a same or separate storage. In addition, the structure of the EOX file is a logical structure and not necessarily a physical structure. Thus, memory storage configured according the described EOX file need to store the file contiguously.

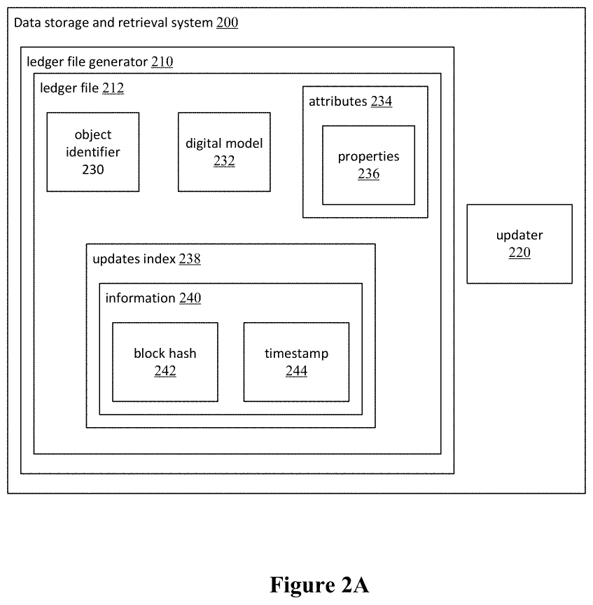

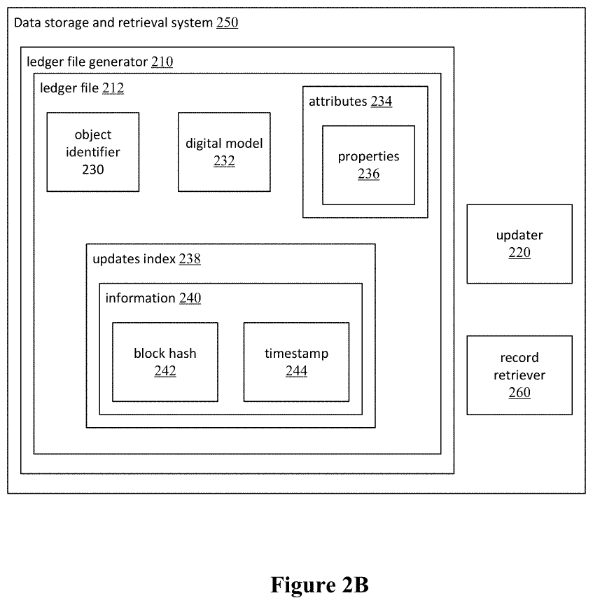

FIGS. 2A and 2B are block diagram that describe example data storage and retrieval systems according to some embodiments of the present disclosure. Data storage and retrieval systems 200 and 250 can have a hardware configuration structured as one or more computers, for example, one or more blade server devices, standalone server devices, personal computers, routers, hubs, switches, bridges, firewall devices, intrusion detection devices, mainframe computers, network-attached storage devices, and other types of computing devices. In this manner, operations for generating and updating a ledger file can be stored on the one or more computers and even in a distributed manner. Similarly, the ledger file and associated content can be stored on the one or more computers and even in a distributed manner. For example, attribute information can be stored in a scalable embedded database. In addition, the operations for generating and updating a ledger file may be performed by one or more processors at one or more of the one or more computers.

Referring to FIG. 2A, a data storage and retrieval system 200 for one or more computers includes a ledger file generator 210 that generates a data structure of a ledger file 212 in the one or more computers and an updater 220 that assigns a corresponding security tag to an updates index 238 for indexing each newly added record of information of an attribute to the ledger file 212. The ledger file 212 generated by the data storage and retrieval system 200 can include the features described with respect to ledger file 100 of FIG. 1A. For example, in some embodiments, the ledger file generator 210 can generate a ledger file 212 including an object identifier 230 that identifies the ledger file 212 for an asset related to a physical entity, a digital model 232 of the asset, and an updates index 238 for storing security tags. The ledger file generator 210 can further generate attributes 234 of the ledger file 212. The attributes 234 include properties 236 providing a record of information about the asset or the detail associated therewith. The updates index 238 includes security tag information 240. The security tag information 240 includes a block hash 242 and a timestamp 244. The block hash 242 identifies a block/location of the updated information. In some cases, the ledger file generator 210 assigns information to a block identified by the security tag (e.g., with block hash 242) assigned by the updater 220.

The ledger file 212 generated by the data storage and retrieval system 200 allows for a means to record data of an entity's development, sale, use, growth, and changes to be recorded in a manner that can easily be converted to information reflected in a virtual reality space or other graphical user interface. The particular activities (and attributes) that the data storage and retrieval system tracks for an asset depends on the particular asset/physical entity. The data storage and retrieval system 200 can add new attributes to an existing ledger file 212 as well as enable updates to data associated with existing attributes.

The file format described above can enable synchronous association of an asset in real-space to a virtual/digital form as things change with the asset over time. The data storage and retrieval system can leverage application programming interfaces of various applications and devices (including those represented as attributes in the ledger file) to receive updates. The updates and communications can be through manual updates (e.g., via a user interface to a platform associated with the data storage and retrieval system or a user interface to an application that communicates with the data storage and retrieval system), sensors, and other devices The accumulated information can be stored in a distributed environment or decentralized environment with each successive update referencing the immediate previous location (e.g., using the security tag/block hash and referencing a prior block on the chain).

The ledger file 212 provides a way to logically group things together in a manner that allows for ease of real-time updating/synchronous updating of information in the real world and the digital world. The updater 220 enables the flexibility that it is not a requirement for the data storage and retrieval system to store the content itself; however, the system can do so and identify the location in a block (e.g., via the security tag).

Referring to FIG. 2B, a data storage and retrieval system 250 for one or more computers includes the features described with respect to data storage and retrieval system 200 and further includes a record retriever 260 that retrieves a particular newly added record of information using the corresponding security tag. Record retriever 260 includes functionality to enable reading and access to blocks of data (i.e., identified by the security tags). Record retriever 260 can include or access authentication credentials, keys, tokens, and other security measures that enable access to the data.

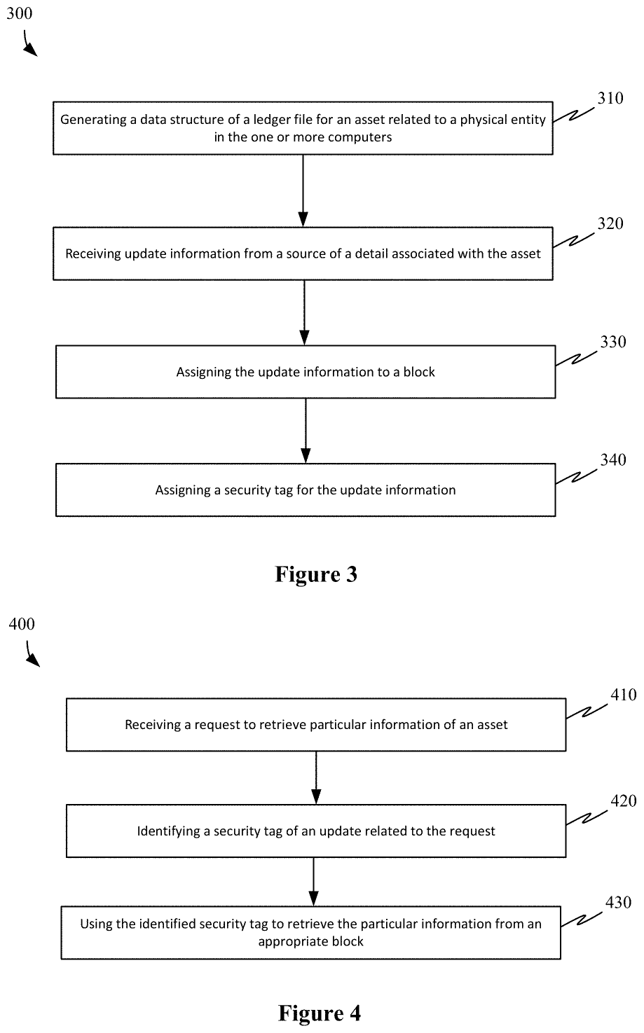

FIG. 3 is a flowchart that describes a method for storing and retrieving data from one or more computers, according to some embodiments of the present disclosure. In some embodiments, the method 300 includes generating (310) a data structure of a ledger file for an asset related to a physical entity. In some embodiments, the method 300 includes receiving (320) update information from a source of a detail associated with the asset; assigning (330) the update information to a block, and assigning (340) a security tag for the update information, the security tag identifying the block. The security tag is assigned to an updates index of the ledger file. A corresponding security tag is assigned to the updates index for indexing each newly added record of information of an attribute to the ledger file. In some cases, the method further includes identifying the source of the detail associated with the asset and storing information of the source of the detail with the security tag for the update information.

FIG. 4 is a flowchart that describes a further method for storing and retrieving data from one or more computers, according to some embodiments of the present disclosure. Referring to FIG. 4, in some embodiments, the method 400 includes receiving (410) a request to retrieve particular information of the asset; identifying (420) a corresponding security tag of an update related to the request; and using (430) the identified security tag to retrieve the particular information from an appropriate block. The request to retrieve the particular information of the object can include the object identifier and a search query. The search query can be about the asset, the physical entity, a particular detail, an attribute of interest, etc. For the most recent information, the timestamp information of the security tag can be used.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts that would be recognized by one skilled in the art are intended to be within the scope of the claims.

What is claimed is:

1. A data storage and retrieval system for one or more computers, comprising:
a ledger file generator that generates a data structure of a ledger file in the one or more computers, the data structure of the ledger file including:
an object identifier that identifies the ledger file for an asset related to a physical entity;
a digital model of the asset;
attributes, each attribute corresponding to a detail associated with the asset related to the physical entity and having properties providing a record of information about the asset or the detail associated therewith; and
an updates index for storing security tags, each security tag having information including a block hash and a timestamp; and
an updater that assigns a corresponding security tag to the updates index for indexing each newly added record of information of an attribute to the ledger file.

2. The data storage and retrieval system for one or more computers of claim 1, wherein the ledger file generator assigns information to a block identified by a particular security tag.

3. The data storage and retrieval system for one or more computers of claim 1, wherein the digital model of the asset comprises resource location information of the digital model.

4. The data storage and retrieval system for one or more computers of claim 1, wherein the digital model of the asset is a 3-D model file, a virtual reality file, an image file, or a document file.

5. The data storage and retrieval system for one or more computers of claim 1, wherein the attributes comprise:
a parent attribute indicating a source of the information or indicative of a category of information; and
one or more child attributes of the parent attribute, the one or more child attributes being related to content associated with the source of information or being indicative of an entity in the category of information.

6. The data storage and retrieval system for one or more computers of claim 5, wherein the parent attribute indicates the source of the information, wherein the source of the information is an application or device.

7. The data storage and retrieval system for one or more computers of claim 1, wherein the attributes comprise attribution information indicating a source or creator of the physical entity.

8. The data storage and retrieval system for one or more computers of claim 1, wherein each security tag further includes what or who is a source of an update.

9. The data storage and retrieval system for one or more computers of claim 8, wherein the source of the update is an application or device.

10. The data storage and retrieval system for one or more computers of claim 1, further comprising a record retriever that retrieves a particular added record of information using the corresponding security tag.

11. A method for storing and retrieving data from one or more computers, comprising:
generating a data structure of a ledger file in the one or more computers, the data structure of the ledger file including:
an object identifier that identifies the ledger file for an asset related to a physical entity;
a digital model of the asset;
attributes, each attribute corresponding to a detail associated with the asset related to the physical entity and having properties providing a record of information about the asset or the detail associated therewith; and
an updates index for storing security tags, each security tag having information including a block hash and a timestamp; and
assigning a corresponding security tag to the updates index for indexing each newly added record of information of an attribute to the ledger file.

12. The method for storing and retrieving data from one or more computers of claim 11, further comprising:
receiving update information from a source of a detail associated with the asset; and
assigning the update information to a block;
wherein the corresponding security tag assigned to the update information at the updates index identifies the block.

13. The method for storing and retrieving data from one or more computers of claim 12, further comprising:
identifying the source of the detail associated with the asset, and
storing information of the source of the detail with the security tag for the update information.

14. The method for storing and retrieving data from one or more computers of claim 11, further comprising:
receiving a request to retrieve particular information of the asset;
identifying a security tag of an update related to the request; and
using the identified security tag to retrieve the particular information from an appropriate block.

15. The method for storing and retrieving data from one or more computers of claim 14, wherein the request to retrieve particular information of the asset comprises the object identifier and a search query.
